(19) United States
(12) Patent Application Publication
(10) Pub. No.: US 2026/0364597 A1
(43) Pub. Date: Nov. 19, 2026

(54) LEVERAGING PHOTOTHERMAL HEATING FOR SURFACE PROCESSING OF ACTIVE AGENTS

(71) Applicant: Trustees of Dartmouth College, Hanover, NH (US)

(72) Inventors: John Molinski, Wilder, VT (US); John X.J. Zhang, Hanover, NH (US)

(73) Assignee: Trustees of Dartmouth College, Hanover, NH (US)

(21) Appl. No.: 19/487,562

(22) PCT Filed: May 24, 2024
(86) PCT No.: PCT/US2024/031001

§ 371 Date: Nov. 25, 2025

Related U.S. Application Data
(60) Provisional application No. 63/469,029, filed on May 25, 2023.

(51) Int. Cl. B01L 3/00

(52) U.S. Cl. CPC B01L 3/502715; B01L 3/502761; B01L 2200/027; B01L 2200/0647; B01L 2300/1861; B01L 2300/1894; B01L 2400/043

(57) ABSTRACT

Embodiments of the present disclosure pertain to methods of loading a material with an active agent by (1) associating the material with a surface; (2) associating the active agent with the material; (3)photothermally heating the surface-associated material to result in the encapsulation of the active agent into the surface-associated material; and (4) dissociating the active agent—14 loaded material from the surface. Additional embodiments of the present disclosure pertain to systems operable for loading a material with an active agent. The systems of the present disclosure may include (1) a surface operable to associate with a material, (2) a potothermal heating source proximal to the surface and operable to heat the surface, and (3) a magnet operable to apply a magnetic field to the surface.

### CROSS-REFERENCE TO RELATED APPLICATIONS

This application claims priority to U.S. Provisional Patent Application No. 63/469,029, filed on May 25, 2023. The entirety of the aforementioned application is incorporated herein by reference.

### BACKGROUND

Current methods of loading active agents into particles have numerous limitations. Numerous embodiments of the present disclosure aim to address the aforementioned limitations.

### SUMMARY

In some embodiments, the present disclosure pertain to methods of loading at least one material with at least one active agent. In some embodiments, the methods of the present disclosure include:
(1) associating the material with a surface; (2) associating the active agent with the material; (3) photothermally heating the surface-associated material to result in the encapsulation of the active agent into the surface-associated material; and (4) dissociating the active agent-loaded material from the surface. In some embodiments where the surface includes a magnetic surface, the active agent loading methods of the present disclosure include: (1) capturing a material by magnetic beads to form a magnetic bead-material complex; (2) associating the magnetic beads with a magnetic surface; (3) applying a magnetic field to the magnetic surface to result in the immobilization of the magnetic beads on the magnetic surface; (4) associating the active agent with the material; (5) photothermally heating the surface-associated material to result in the encapsulation of the active agent into the surface-associated material; (6) removing the magnetic field from the magnetic surface to result in the dissociation of the active agent-loaded material from the magnetic surface; and (7) dissociating the active-agent loaded material from the magnetic beads.

Additional embodiments of the present disclosure pertain to systems operable for loading a material with an active agent. The systems of the present disclosure generally include a surface operable to associate with a material, and a photothermal heating source proximal to the surface and operable to heat the surface. In some embodiments, the systems of the present disclosure also include a magnet operable to apply a magnetic field to the surface.

### DRAWINGS

FIG. 1A illustrates a method of loading materials with one or more active agents in accordance with various embodiments of the present disclosure.

FIG. 1B illustrates a method of utilizing a magnetic surface to load materials with one or more active agents in accordance with various embodiments of the present disclosure.

FIG. 1C illustrates a method of utilizing a magnetic surface to load exosomes with one or more active agents in accordance with various embodiments of the present disclosure.

FIG. 1D illustrates the formation of magnetic beads for use in loading exosomes with one or more active agents.

FIG. 1E illustrates a system operable for loading materials with one or more active agents in accordance with various embodiments of the present disclosure.

FIGS. 2A-2E illustrate the design and validation of a photothermal engineering system. FIG. 2A shows the design of a photothermal engineering platform, which consists of a removable housing for magnetic and magnetic microchip, a light emitting diode (LED), and a thermoelectric cooler (TEC) to provide active cooling. FIG. 2B provides a schematic showing the principle of magnetophoretic capture using flow-invasive micromagnets (bottom) and photothermal heating of patterned micromagnets to enable exosome engineering (top). FIGS. 2C-2E show heatmaps highlighting full system output power (FIG. 2C), binned output levels used for testing (FIG. 2D), and level of loading of doxorubicin at each binned level (FIG. 2E) using fluorescence intensity.

FIGS. 3A-3C illustrate matrix-based photoporation mechanisms. Schematics show traditional photoporation (FIG. 3A), nanoparticle-mediated photoporation (FIG. 3B), and proposed matrix-based photoporation (FIG. 3C). Upon irradiation, traditional photoporation (FIG. 3A) relies on the rapid localized heating to induce temporary disruption of the biomolecular membrane, depicted here as a dotted line. Nanoparticle-mediated photoporation (FIG. 3B) increases localized heating and efficiency of photoporation. For matrix-based photoporation (FIG. 3C), Applicant hypothesizes rapid localized heating, which induces efficient disruption of biomolecular membranes.

FIGS. 4A-4G illustrate a system optimization and proof-of-concept using human colon carcinoma (COLO-1) derived exosome standards. FIG. 4A provides a schematic showing an immunomagnetic capture and engineering process. FIGS. 4B-4C show doxorubicin (DOX) loading amount using fluorescence intensity as proxy at each binned power output, for unpatterned (FIG. 4B) and patterned (FIG. 4C) magnetic microchips. FIG. 4D shows aggregated results comparing DOX loading within unpatterned and patterned microchips (P<0.05). FIGS. 4E-4F show system optimization for DOX loading, including number of 50 ms pulses (FIG. 4E) and pulse duration (FIG. 4F). FIG. 4G shows the 3D structure of doxorubicin with a maximum diameter and chemical formula.

FIGS. 5A-5I show data related to small molecule exosome loading. FIGS. 5A-5D show 100× confocal microscope images in differential interference contrast (FIG. 5A), DOX channel (Ex. 470/Em. 595) (FIG. 5B), DiD channel (Ex. 644 nm. Em. 663 nm) (FIG. 5C), and combined DOX and DiD (FIG. 5D). Scale bars are 1 μm. FIG. 5E shows exosome counts loaded with DiD membrane dye as control and DOX as actively loaded compound, showing a ~97% permeation efficiency within exosomes. FIG. 5F shows optimized DOX loading formulation compared to the double negative control (no patterning and no photothermal heating, P<0.05). FIGS. 5G-5I show 20× confocal images of magnetic patterning in brightfield (FIG. 5G), DiD channel (FIG. 5H), and DOX channel (FIG. 5I), showing successful loading of DOX within exosomes captured, purified, and engineered directly from patient plasma samples. Scale bars are 200 μm.

FIGS. 6A-6I show cargo agnostic loading with an EXOtherm platform. Schematics show cargo loaded within exosomes including doxorubicin (FIG. 6A), Cy3-labeled siRNA (FIG. 6B), and Cas9-GFP (FIG. 6C), along with corresponding molecular weights. FIGS. 6D-6I show confocal microscopy images of exosomes captured upon magnetic microchips for doxorubicin (FIGS. 6D and 6G), Cy3-labeled siRNA (FIGS. 6E and 6H), and Cas9-GFP (FIGS. 6F and 6I). All scale bares are 200 μm.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Current methods of loading active agents into particles have numerous limitations. For instance, such methods are time intensive and designed for specific starting materials. As such, existing methods of loading active agents into particles offer limited adaptability. Moreover, such existing methods introduce significant room for sample loss, batch-to-batch heterogeneity, and error.

For instance, due to their native functions within cell-to-cell communication, exosomes provide an ideal scaffold for delivering biotherapeutics into cells. However, current exosome biotherapeutic manufacturing approaches are time intensive. In particular, such manufacturing approaches utilize cell-line engineering and batch-based culture, which in turn offer limited adaptability.

Moreover, current methods for exosome isolation, purification, and cargo loading require multiple days of processing time, provide low yield (~4% process yield), and require numerous discrete processing steps (often >10). Such limitations introduce significant room for sample loss, batch-to-batch heterogeneity, and error.

For exosome isolation and purification, bioreactor-based exosome production from a single cell line is typically employed, followed by a series of specialized multistage chromatography and filtration processes, respectively. Such steps not only induce significant process complexity, but each compounds the risk of altering biological properties critical to therapeutic delivery and function.

Exosome cargo loading methods are especially nascent in their development and at present often rely on cell-line engineering or simple incubation steps, with the intention of fusing cargo to the exosome surface upon biogenesis or diffusion into the membrane, respectively. Such processing inefficiencies result in low loading efficiencies for many therapeutically desirable classes.

In addition to these processing inefficiencies and a highly discretized workflow, biotherapcutic manufacturing pipelines are largely static, thereby requiring specific attention and specialization for each cell line or therapeutic cargo class. Such rigidity is especially problematic in the era of personalized medicine.

Towards isolation and purification, microfluidic systems have been introduced, offering selective enrichment of exosome populations through affinity, chemical, electrical, or mechanical means, with varying levels of efficiency, throughput, and performance. Likewise, towards cargo loading, an array of techniques, including freeze/thaw cycles, electroporation, sonication, incubation, and nanomaterial-mediated approaches have been explored, which have yet to yield a preeminent path forward.

As such, a need exists for improved methods and systems for loading active agents into particles. For instance, to realize the potential of exosomes within biotherapeutic applications, a need exists for improved methods and systems for exosome isolation and active agent loading. Numerous embodiments of the present disclosure aim to address the aforementioned limitations.

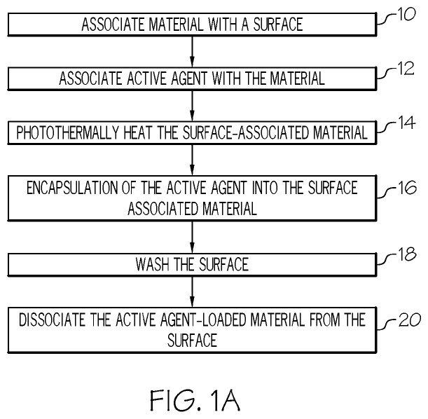

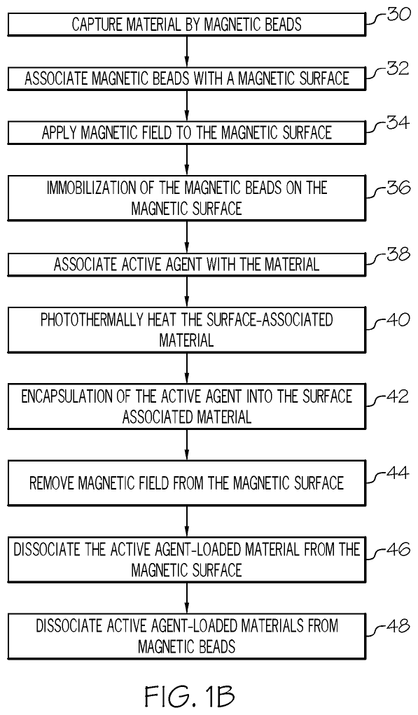

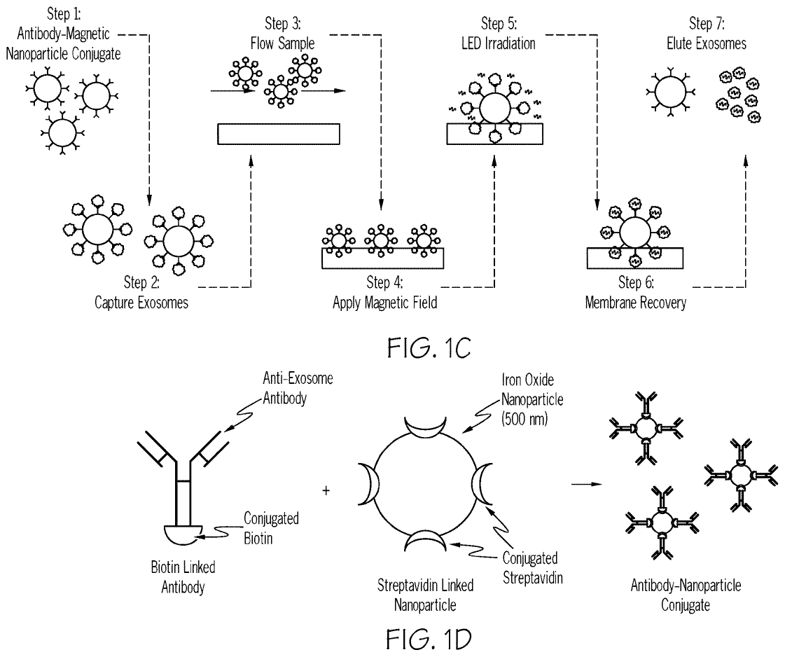

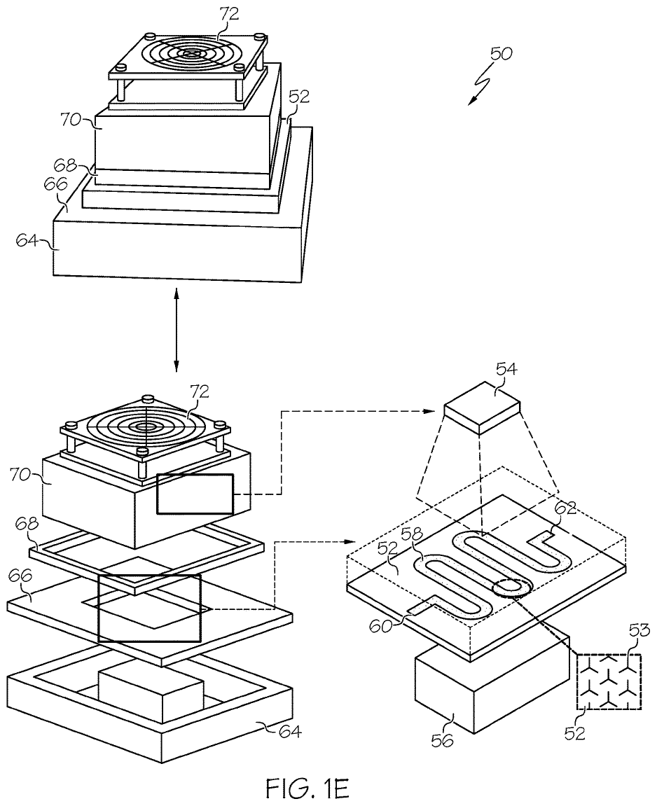

In some embodiments, the present disclosure pertain to methods of loading at least one material with at least one active agent. In some embodiments illustrated in FIG. 1A, the methods of the present disclosure include: associating the material with a surface (step 10); associating the active agent with the material (step 12); photothermally heating the surface-associated material (step 14) to result in the encapsulation of the active agent into the surface-associated material (step 16); and dissociating the active agent-loaded material from the surface (step 20). In some embodiments, the methods of the present disclosure also include a step of washing the surface prior to dissociating the active agent-loaded material from the surface (step 18).

In some embodiments, the surface includes a magnetic surface. In some embodiments, illustrated in FIG. 1B, the active agent loading methods of the present disclosure include: capturing a material by magnetic beads to form a magnetic bead-material complex (step 30); associating the magnetic beads with a magnetic surface (step 32); applying a magnetic field to the magnetic surface (step 34) to result in the immobilization of the magnetic beads on the magnetic surface (step 36); associating the active agent with the material (step 38); photothermally heating the surface-associated material (step 40) to result in the encapsulation of the active agent into the surface-associated material (step 42); and removing the magnetic field from the magnetic surface (step 44) to result in the dissociation of the active agent-loaded material from the magnetic surface (step 46). In some embodiments, the methods of the present disclosure also include a step of dissociating the active-agent loaded material from the magnetic beads (step 48).

A more specific example of an active agent loading method of the present disclosure is illustrated in FIGS. 1C-1D, which pertains to a method of loading active agents into exosomes through the use of a magnetic surface and antibody-magnetic nanoparticle conjugates that are specific for capturing exosomes (FIG. 1D). As illustrated in FIG. 1C for this example, exosomes are first captured by antibody-magnetic nanoparticle conjugates to form a magnetic nanoparticle-exosome complex (steps 1-2). Thereafter, the magnetic nanoparticle-exosome complexes are associated with a magnetic surface by flowing the complexes through the magnetic surface (step 3). Next, a magnetic field is applied to the magnetic surface to result in the immobilization of the magnetic beads on the magnetic surface (step 4). Active agents are then associated with the immobilized exosomes and photothermally heated (step 5) to result in the encapsulation of the active agent into the immobilized exosomes (step 6). Next, the magnetic field is removed from the magnetic surface to result in the dissociation of the immobilized active agent-loaded exosomes from the magnetic surface. Finally, the active-agent loaded exosomes are dissociated from the antibody-magnetic nanoparticle conjugates (step 7).

Additional embodiments of the present disclosure pertain to systems operable for loading a material with an active agent. For illustrative purposes, the systems of the present disclosure may be depicted as system 50 in FIG. 1E. System 50 generally includes a surface 52 operable to associate with a material, and a photothermal heating source 54 proximal to surface 52 that is operable to heat surface 52.

In some embodiments, photothermal heating source 54 is positioned above surface 52. In some embodiments, surface 52 is patterned with a plurality of patterns 53. In some embodiments, surface 52 is also in the form of a microfluidic channel 58, which may include an inlet 60 for receiving a material and an active agent, and an outlet 62 for releasing active agent-loaded materials. In some embodiments, microfluidic channel 58 is in encapsulated form.

In some embodiments, system 50 also includes a magnet 56 operable to apply a magnetic field to surface 52. In some embodiments, magnet 56 is positioned below surface 52.

In some embodiments, system 50 also includes a housing unit 64 operable for housing magnet 56. In some embodiments, housing unit 64 is operable to control spacing between magnet 56 and surface 52 to ensure consistent magnetization of surface 52.

In some embodiments, system 50 also includes a housing unit 66 operable for housing surface 52, a housing unit 70 operable for housing photothermal heating source 54, and a cooling unit 72 operable for cooling photothermal heating source 54. In some embodiments, cooling unit 72 is in the form of a thermoelectric cooler.

As set forth in more detail herein, the methods and systems of the present disclosure can have numerous embodiments. As also set forth in more detail herein, the methods and systems of the present disclosure can have numerous advantages and applications.

### Materials

The methods and systems of the present disclosure may be utilized to load active agents into various materials. For instance, in some embodiments, the materials include, without limitation, particles, magnetic particles, nanoparticles, magnetic nanoparticles, polymeric particles, viral particles, virus-like particles, lipid-based particles, nanomaterials, magnetic nanomaterials, materials that include a biomolecular membrane, exosomes, liposomes, cells, or combinations thereof.

In some embodiments, the materials include materials that include a biomolecular membrane. In some embodiments, the materials include exosomes. In some embodiments, the materials include cells. In some embodiments, the cells include, without limitation, immune cells, T-cells, B-cells, or combinations thereof. In some embodiments, the cells include exosomes of varying cellular origins. In some embodiments, the exosome origin includes, without limitation, exosomes from T-cells, B-cells, mesenchymal stem cells, plasma-derived exosomes, or combination thereof. In some embodiments, the materials are derived from cell culture mediums. In some embodiments, the materials are derived from patient biofluids including plasma, whole blood, urine, cerospinal fluid (CSF), or combinations thereof.

### Active Agents

The methods and systems of the present disclosure may be utilized to load various active agents into materials. For instance, in some embodiments, the active agents include, without limitation, drugs, small molecules, biologics, proteins, peptides, peptoids, nucleic acids, RNA, siRNA, mRNA, miRNA, DNA, genes, gene editing systems, clustered regularly interspaced short palindromic repeats (CRISPR)-Cas gene editing systems, image contrast agents, quantum dots, or combinations thereof. In some embodiments, the active agents include drugs. In some embodiments, the active agents include a gene editing system that is operable to insert a gene into a cell. In some embodiments, the active agents include a gene for expression in a cell.

### Association of an Active Agent With a Material

The methods of the present disclosure may associate active agents with materials in various manners. For instance, in some embodiments, the association of active agents with materials occurs prior to associating the materials with a surface. In some embodiments, the association of active agents with materials occurs during the association of the materials with a surface. In some embodiments, the association of active agents with materials occurs after associating the materials with a surface.

The association of active agents with materials can occur in various manners. For instance, in some embodiments, the association of the active agents with materials occurs at room temperature. In some embodiments, the association of active agents with materials includes incubating the materials and active agents. In some embodiments, the association of active agents with materials includes mixing the materials and the active agents in a liquid.

### Surfaces

The methods and systems of the present disclosure may utilize various surfaces for loading active agents into materials. For instance, in some embodiments, the surface is in the form of a membrane that includes a plurality of photosensitizers. In some embodiments, the photosensitizers arc operational to induce localized heating of the membrane upon photothermal heating. In some embodiments, the photosensitizers include magnetic nanoparticles. In some embodiments, the membrane is in the form of an elastomeric matrix.

In some embodiments, the surfaces of the present disclosure include a patterned surface. In some embodiments, the patterned surface is operational for facilitating the association of a material with the surface. The surfaces of the present disclosure may include various patterns. For instance, in some embodiments, the patterned surface includes one or more shapes. In some embodiments, the shapes include, without limitation, squares, herringbones, Y-like shapes (e.g., Y-like shapes 53 of surface 52 in FIG. 1E), X-like shapes, or combinations thereof. In some embodiments, the patterned surface can be encapsulated within a microfluidic channel. In some embodiments, the patterning can be rationally designed to influence a native fluidic environment. In some embodiments, the patterning can be rationally designed to influence the microscale magnetic field. In some embodiments, the pattern includes one or more shapes. In some embodiments, the height of the patterned surface can be changed, from a range of 1 μm to 200 μm.

In some embodiments, the surfaces of the present disclosure include material binding agents that are operational for associating a material with a surface. In some embodiments, the material binding agents include, without limitation, antibodies, aptamers, peptides, peptoids, small molecules, or combinations thereof.

In some embodiments, the surfaces of the present disclosure are in the form of a microfluidic channel (e.g., microfluidic channel 58 shown in FIG. 1E). In some embodiments, the microfluidic channel includes an inlet for receiving a material and an active agent (e.g., inlet 60 shown in FIG. 1E), and an outlet for releasing an active agent-loaded material (e.g., outlet 62 shown in FIG. 1E). In some embodiments, the microfluidic channel is in encapsulated form.

In some embodiments, the surfaces of the present disclosure include a magnetic surface. In some embodiments, the magnetic surface includes a plurality of magnetic particles. In some embodiments, the magnetic particles include, without limitation, magnetites, iron oxide particles, Fe₃O₄-containing particles, γ-Fe₂O₃-containing particles, or combinations thereof. In some embodiments, the plurality of magnetic particles is encapsulated within a polymer matrix. In some embodiments, the plurality of magnetic particles is encapsulated within an elastomer matrix. In some embodiments, the weight loading percentage of the magnetic particles within a matrix can range from 1% to 75% weight loading. In some embodiments, the magnetic surface is associated with a magnet operational to apply a magnetic field to the magnetic surface (e.g., magnet 56 shown in FIG. 1E).

### Associating of Materials With Surfaces

The methods of the present disclosure may associate materials with surfaces in various manners. For instance, in some embodiments, the association of materials with surfaces includes flowing materials through the surface. In some embodiments, the association of materials with surfaces includes incubating materials with a surface.

In some embodiments, the association of materials with a surface includes applying a magnetic field to a magnetic surface. In some embodiments, the association of materials with a magnetic surface includes: (1) capturing materials by magnetic beads to form magnetic bead-material complexes; (2) associating the magnetic beads with the magnetic surface; and (3) applying a magnetic field to the magnetic surface to result in the immobilization of the magnetic bead-material complex on the magnetic surface. In some embodiments, the association of magnetic beads with a magnetic surface occurs after the capture of materials by magnetic beads. In some embodiments, the association of magnetic beads with a magnetic surface occurs before the capture of materials by the magnetic beads. In some embodiments, the association of magnetic beads with a magnetic surface occurs during the capture of materials by magnetic beads.

In some embodiments, the magnetic beads include magnetic particles and material binding agents on the magnetic particles. In some embodiments, material capture includes the capture of materials by the material binding agents. In some embodiments, the material binding agents include, without limitation, antibodies, aptamers, peptides, peptoids, small molecules, or combinations thereof.

### Photothermal Heating of Surface-Associated Materials

Photothermal heating generally refers to the utilization of photons to heat surface-associated materials. The methods of the present disclosure may photothermally heat surface-associated materials in various manners. For instance, in some embodiments, photothermal heating includes photothermally heating a surface. In some embodiments, the photothermal heating of the surface results in the heating of the material by the surface. In some embodiments, the photothermal heating results in the photoporation of the material.

In some embodiments, the photothermal heating of a surface-associated material includes light irradiation from a light source. In some embodiments, the light source includes a laser. In some embodiments, the light source includes a light emitting diode (LED). In some embodiments, the light source is positioned above a surface during photothermal heating.

In some embodiments, the photothermal heating of a surface-associated material occurs at sequential intervals. In some embodiments, each interval lasts from about 5 ms to about 250 ms. In some embodiments, each interval is separated by at least 5 ms. In some embodiments, the time of each interval is separated by an equal time between. In some embodiments, the interval time is longer than the time between. In some embodiments, the interval time is shorter than the time between. In some embodiments, the photothermal heating occurs during at least 3 sequential intervals. In some embodiments, the photothermal heating occurs with more sequential intervals, such as up to 10 sequential intervals. In some embodiments, only a single photothermal heating interval is used. In some embodiments, each interval within a sequence has a different time duration. In some embodiments, each interval has a different power density. In some embodiments, each interval has the same power density.

The methods and systems of the present disclosure may utilize various photothermal heating sources. For instance, in some embodiments, the photothermal heating source includes a light source operable to emit light irradiation. In some embodiments, the light source includes a laser. In some embodiments, the light source includes a light emitting diode (LED). In some embodiments, the light source includes a Chip-On-Board (COB) LED. In some embodiments, the light source includes a Surface-Mounted Device (SMD) LED. In some embodiments, the light source includes a Multiple-Chips-On-Board (MCOB) LED. In some embodiments, the wavelength of the light source corresponds to the absorbance of the patterned materials. In some embodiments, the wavelength of the light source corresponds to the absorbance of the surface-associated materials. In some embodiments, the wavelength is within the visible regime (380-700 nm). In some embodiments, the wavelength in is the near-IR regime (800-2500 nm).

### Washing of Surface-Associated Materials

In some embodiments, the methods of the present disclosure also include a step of washing a surface-associated material. In some embodiments, the washing step occurs prior to dissociating active agent-loaded materials from a surface. In some embodiments, the washing step separates unloaded active agents from surface-associated and active agent-loaded materials. In some embodiments, the washing step includes flowing a wash buffer through a surface. In some embodiments, the wash buffer includes phosphate-buffered saline (PBS).

### Dissociation of Active Agent-Loaded Materials From a Surface

In some embodiments, the methods of the present disclosure also include a step of dissociating active-agent loaded materials from a surface. For instance, in some embodiments, the dissociation step includes a step of flowing an elution buffer through the surface. In some embodiments, the elution buffer includes a Tris-Cl buffer.

In some embodiments where a surface includes a magnetic surface, the dissociation step includes a step of removing a magnetic field from the magnetic surface to result in the dissociation of active agent-loaded materials from the magnetic surface. In some of such embodiments, the active agent-loaded materials may be immobilized on the magnetic surface through magnetic beads that form magnetic bead-material complexes on a magnetic surface.

### ADVANTAGES AND APPLICATIONS

The methods and systems of the present disclosure can have various advantages. For instance, in some embodiments, the methods of the present disclosure may be utilized to load various active agents into various materials in a consistent, facile, cost effective and adaptable manner.

As such, the methods and systems of the present disclosure can have various applications. For instance, in some embodiments, the materials include T-cells and the active agents include one or more genes encoding a T-cell receptor. In such embodiments, the methods of the present disclosure may be used to engineer chimeric antigen receptor T-cells for various therapeutic applications.

In some embodiments, the materials include exosomes and the active agents include one or more drugs. In some of such embodiments, the methods of the present disclosure may be used to engineer therapeutic exosomes. In some of such embodiments, the methods of the present disclosure may be used to engineer exosomes with diagnostic potential. In some embodiments, the methods of the present disclosure may be used to engineer exosomes with theranostic potential. In some embodiments, the methods of the present disclosure may be used to engineer exosomes for delivery to specific or single cell types. In some embodiments, the methods of the present disclosure may be used to engineer exosomes for delivery to multiple cell types at once. In some of such embodiments, the methods of the present disclosure may be used to engineer exosomes which can then engineer cells. In some embodiments, the engineered exosomes can then be used to deliver active agents to cells.

### ADDITIONAL EMBODIMENTS

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### Example 1. Rapid and Scalable Screening and Drug Loading Within Patient-Derived Exosomes for Autologous Therapies

In this Example, Applicant reports the development of EXOtherm, a microtechnology-based system for the capture and engineering of autologous exosomes using magnetic microchips and photothermal heating, respectively. Through on-chip photoporation, Applicant demonstrates tunable exosomal loading with the small molecule chemotherapeutic drug doxorubicin (DOX) and have optimized system parameters (i.e., doses and dose rate) to maximize loading and permeation efficiency. With optimized parameters, Applicant has demonstrated loading of exosomes isolated directly from patient plasma samples, highlighting the potential of the EXOtherm for the development of autologous therapies. To elucidate the cargo agnostic nature of this platform, Applicant also demonstrates the loading of labile cargo, including nucleic acids (siRNA) and proteins (Cas9), molecular classes of significant therapeutic interest. As such, EXOtherm makes significant steps towards enabling integrated immunomagnetic exosome isolation and cargo agnostic photothermal loading within a modular benchtop system.

### Example 1.1. Exotherm Platform for Integrated Exosome Isolation and Cargo Loading

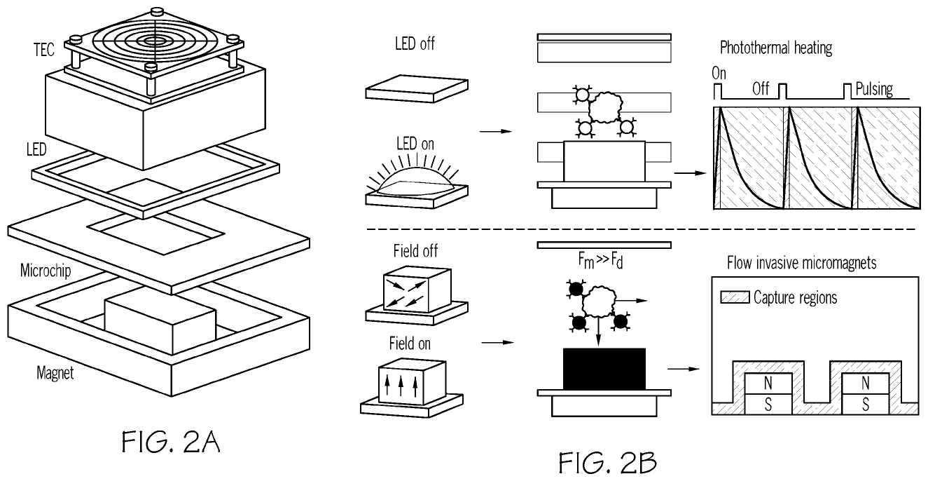

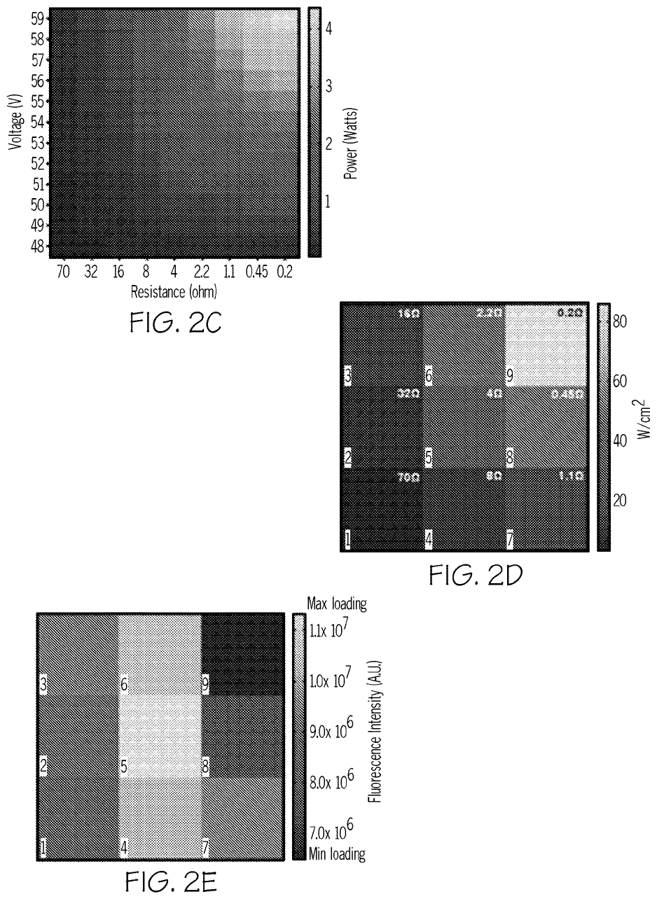

As illustrated in FIGS. 2A-2B, the EXOtherm platform can be broken down into two main components, namely (1) magnetic microchips, and (2) a photothermal engineering system. Magnetic microchips are fabricated using a scalable laser-based fabrication developed by Applicant's group that is suitable for rapid and scalable microchip fabrication and enables efficient immunomagnetic exosome sorting via flow-invasive micromagnets which generate high gradient fields throughout the biofluid medium. Surrounding this main component, Applicant has designed and developed a photothermal engineering system, EXOtherm, that enables integrated on-chip cargo loading within captured exosomes and fits within a minimal form factor enabling benchtop use (FIG. 2A).

EXOtherm consists of a 3D-printed housing included of three interconnected components: the base, microchip holder, and thermoelectric cooler (TEC) holder, plus a separate LED module and programmable power supply (FIG. 2A). The base harbors a permanent neodymium magnet which magnetizes the patterned micromagnets whereas the microchip holder controls spacing between the permanent magnet and magnetic microchip ensuring consistent magnetization. Magnetic microchips are aligned and affixed in place within the holder by magnetic clips which can easily be removed following processing to release the microchip.

Situated above, the TEC holder controls the spacing between magnetic microchips and the LED module, ensuring consistent and repeatable sample irradiation. To promote ease of use, each component, including the microchip and TEC holder, are connected to one another with magnetic fixtures that allow for modular assembly and rapid sample processing.

Considering the large area patterning feasible within magnetic microchips, Applicant sought to enable efficient large-area engineering and as such rather than a laser source chose to utilize a high-power chip-on-board LED (Luminus, CLM-22) for irradiation. Separate from the 3D-printed housing and LED module, a programmable power supply (Keysight 36155A) provides fine-tuned control over optical dose, and tunability in pulse width, duration, and number of pulses.

Optical power density is controlled via interchangeable chassis mount resistors and preset voltages on the programmable power supply. Upon optical characterization, EXOtherm showed precise control dose control from 0.1 W/cm²-100 W/cm² (FIGS. 2C-2E), pulse durations as short as 20 ms, and reliable and repeatable dose control, enabling high levels of tunability. The microchip holder and LED module were positioned such that irradiation occurred at a fixed and known distance (5.13 mm), and all optical recordings were measured at this distance to ensure consistency (FIG. 2B).

### Example 1.2. Nanoscale Photoporation for Exosome Cargo Loading

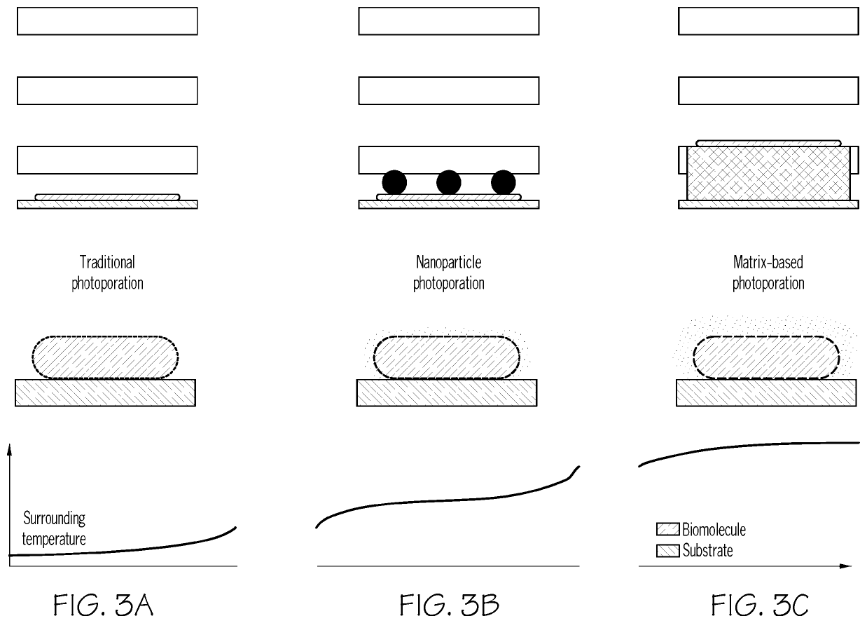

The achieve exosome cargo loading, Applicant developed a novel nanoscale photoporation technique induced by photothermal heating of patterned micromagnets via LED irradiation. Mechanistically, photoporation relies on rapid localized heating, which induces temporary and reversible membrane poration within nearby biomolecules, allowing for the transfection of cargo (FIG. 3A). To increase selectivity and efficiency of this process, nanoparticle sensitizers have been explored, which when bound to biomolecules, act to localize heating (FIG. 3B).

Various nanoparticle formulations, including gold, carbon, and polydopamine-based nanoparticles have been explored, due to their favorable optical properties and biocompatibility. Studies have demonstrated that size of nanoparticle, nanoparticle material, and optical configurations can all play significant roles in transfection efficiency and biomolecule viability, and more importantly that often nanoparticles sensitizers can be difficult from solution following incorporation. As such, efforts have been made to develop methods towards transient photoporation by imbedding photosensitizers within matrices with promising results.

To date, however, photoporation methods have been largely confined to cellular biomolecules, due to the need for specific localization of nanoparticles, or the sparse, uncontrolled distribution of nanoparticles within a matrix. Applicant hypothesized that, through optimized system design and implementation, photoporation could be scaled to the nanoscale to enable the transfection of exosomes. As such, Applicant explored the use of magnetic microchips as a mediator for exosome photoporation (FIG. 3C).

Micromagnets are included of a dense (50% wt/wt) and homogeneous packing of magnetic nanoparticles (~150 nm) within an elastomer matrix. Optical characterization has shown an absorption maximum at ~640 nm, suggesting that micromagnets could act as matrix-based photosensitizers (FIG. 3C). Of further benefit, this microchip architecture was designed to enable robust patterning of magnetically tagged exosomes, which promotes localization of exosomes near photosensitive micromagnets. Taken together, this microchip provides an ideal substrate upon which photothermal engineering of exosomes can occur.

Prior studies demonstrating cellular photoporation have demonstrated the importance of processing parameters (i.e. power density, number of pulses, duration of pulse) on photoporation efficiency. Thus, in the design of the EXOtherm platform, Applicant emphasized modularity and flexibility with each chosen component, potentially critical for optimization within exosome engineering.

### Example 1.3. Small Molecule Loading Using EXOtherm

As an initial proof of concept for EXOtherm, Applicant chose to undertake small molecule loading using doxorubicin hydrochloride, a first line cancer chemotherapy drug for multiple cancers. Doxorubicin represents an ideal testing molecule for platform optimization given its low molecular weight and size, intrinsic stability, and its natural fluorescence that enables facile characterization. Doxorubicin hydrochloride (Sigma) was kept at a stock concentration of 1 mM and within loading tests diluted to a concentration of 150 μM. Characterization of loading magnitude was completed by measuring doxorubicin fluorescence (Ex/Em 470 nm/595 nm) upon a plate reader (Molecular Devices SpectraMax Paradigm). To remove potential heterogeneity resulting from in-house exosome isolation, exosome standards (COLO-1, Novus Biologics) reconstituted in molecular grade water according to manufactures specifications were utilized for initial optimization testing.

Prior to introduction within magnetic microchips, exosomes were magnetically tagged with streptavidin-coated magnetic nanoparticles (MGB magnetic beads, 500 nm, Luna Nanotech) conjugated with biotinylated antibodies (BioLegend) targeting pan-exosome markers CD9, CD63, and CD81. Previous efforts from Applicant's group have optimized the antibody to magnetic bead ratio to maximize conjugation efficiency, which was adopted within this Example.

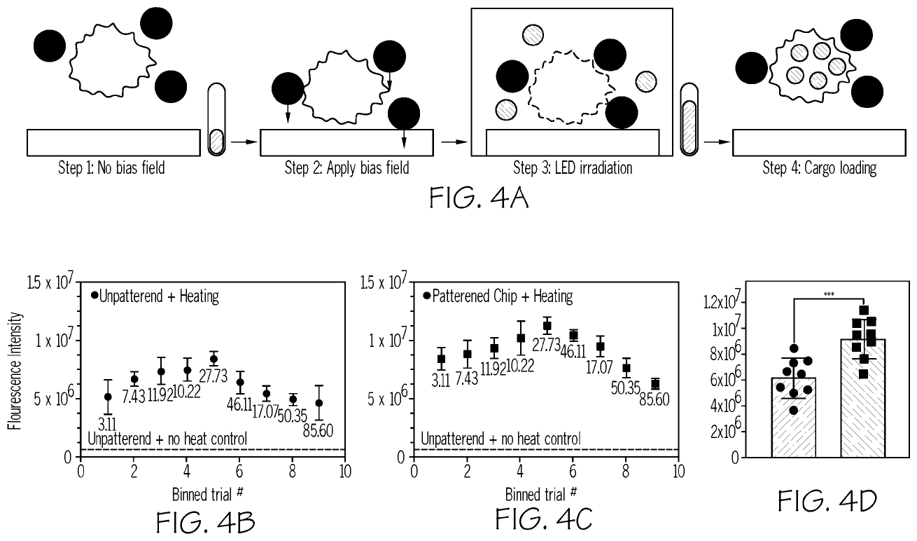

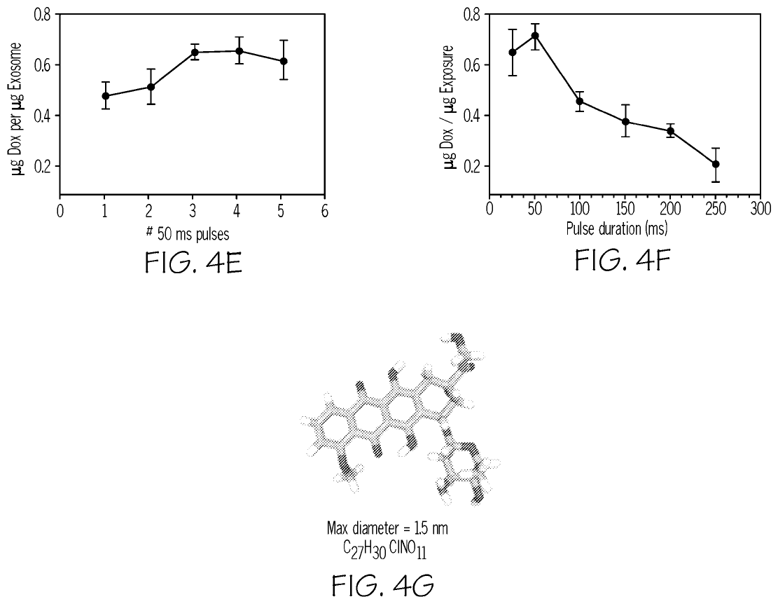

As illustrated in FIG. 4A, doxorubicin loading optimization tests within EXOtherm were performed as follows: A solution (200 μL working volume) containing magnetically tagged exosomes (1 μg exosomes) is combined with DOX and this solution is immediately transferred onto the magnetic microchip. Once affixed within the microchip holder and positioned above the permanent magnet, exosomes are rapidly patterned upon micromagnets. Concurrently, the TEC and LED module are positioned and secured above the microchip, where LED irradiation commences to initiate nanoscale photothermal heating. Following irradiation, still-captured exosomes are rigorously washed to remove residual unloaded DOX. The exosomes are then removed from the microchip holder and washed thoroughly to release exosomes. Loading optimization was completed across the full operational range of the EXOtherm system, by binning the available power range into nine binned and distributed power outputs, with a 50 ms irradiation pulse.

Results from the power density testing are shown in FIGS. 4B-4G, for unpatterned and patterned microchips, with an accumulated comparison comparing each shown in FIG. 4D. The results highlight the benefit of matrix-based photosensitizers. Unpatterned microchips represent traditional photoporation and reflect the contribution of LED irradiation alone, or minor heating resulting from LED irradiation to capture magnetic particles.

With an optimized power density, to further optimize loading, Applicant investigated alterations to pulse duration and number of pulses. Within traditional photoporation using laser-based sources, increased pulse duration leads to higher accumulated doses, eventually leading to irreversible membrane damage and reduced cellular viability. To explore this relationship for exosomes, Applicant selected pulse durations of 20, 50, 100, 150, 200, and 250 ms, and performed loading experiments as completed previously, data from which can be seen in FIGS. 4E-4F.

Similar to cellular biomolecules, with increasing pulse duration, Applicant observed a precipitous drop off in DOX loading amount, which Applicant attributes to a removal of DOX upon washing without exosomal encapsulation. Lastly, Applicant explored the influence of a number of pulses of DOX loading and utilized an equal on/off time of the optimized 50 ms pulse for 1, 2, 3, 4, and 5 pulses.

The rationale behind pulsed operation is that the membrane is given time to recover. Thus, the risk for irreversible disruption is lowered while enabling increased loading efficiency. Results from this study are shown in FIG. 4E, which demonstrates a maximum loading with 3 pulses, followed by insignificant changes with further increases. Taken together, Applicant defined an optimized pulsing scheme (27.73 W/cm² power, 50 ms pulses, and 3 pulses) that was used for all subsequent testing and with it achieved a maximum loading of ~700 ng DOX/μg cxosome (FIG. 4F).

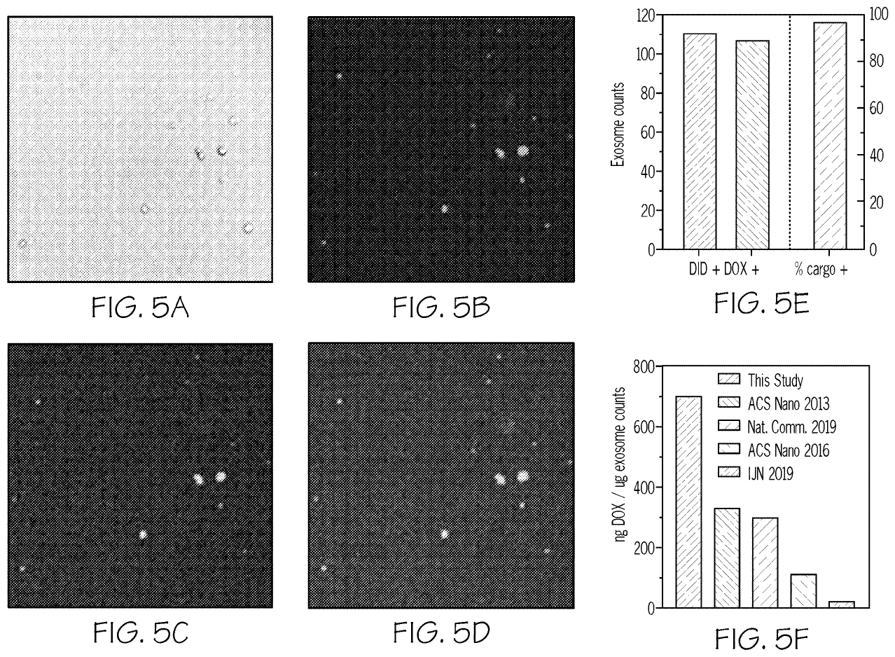

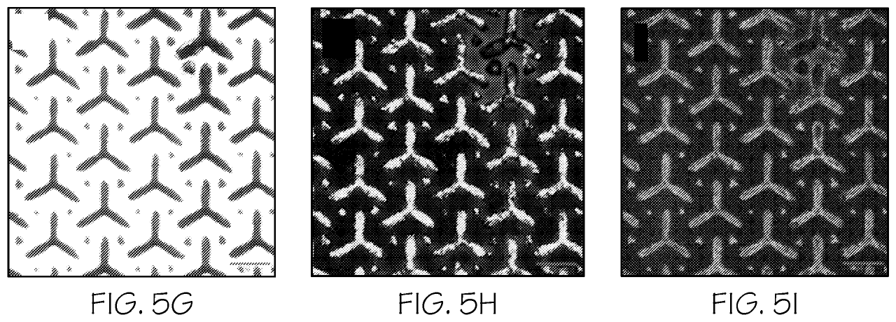

To visual co-localization of cargo within exosomes, a membrane stain (DiD, Invitrogen) was incorporated via incubation to establish a ground truth for exosome location. On-chip imaging was completed using confocal microscopy (Andor W1 Spinning Disc) with doxorubicin excitation completed using a 488 nm laser and DiD excitation via 637 nm laser lines (FIGS. 5A-5D). Subsequent image analysis allowed for the determination of areas where DiD and DOX fluroscence overlapped, and using this Applicant calculated permeation efficiency, defined as the # of DOX+ exosomes/#of DiD+ exosomes (FIGS. 5E-5F).

Results from multiple imaging ROI were combined, leading to an aggregated exosome count of >100, which established a permeation efficiency of ~97%. At this stage, for initial optimization, all testing had been completed using exosome standards within idealized solutions.

Thus, to further elucidate the potential of the EXOtherm platform, Applicant sought to explore the feasibility of capturing and loading exosomes directly from undiluted plasma samples. Plasma was collected, and 200 μL was incubated with capture magnetic nanoparticles and DOX before photothermal loading with optimized parameters. On-chip imaging confirmed DOX loading and equivalent permeation efficiency to Applicant's controlled workflow validation studies, highlighting the potential for the EXOtherm platform to enable the use of exosome from diverse origins (FIGS. 5G-5I).

### Example 1.4. Loading of Labile Molecular Cargo

Having demonstrated small molecule loading within exosomes, Applicant explored the potential of the EXOtherm platform's cargo agnostic loading with the loading of labile molecular cargo. The loading of multiple classes of molecular cargo, such as those with varying molecular weights, charges, and sizes, remains challenging for most carrier modalities (e.g., lipid nanoparticles, polymer nanoparticles, etc.). Utilizing native carriers such as exosomes imposes further challenges, given the numerous challenges that precede their utility.

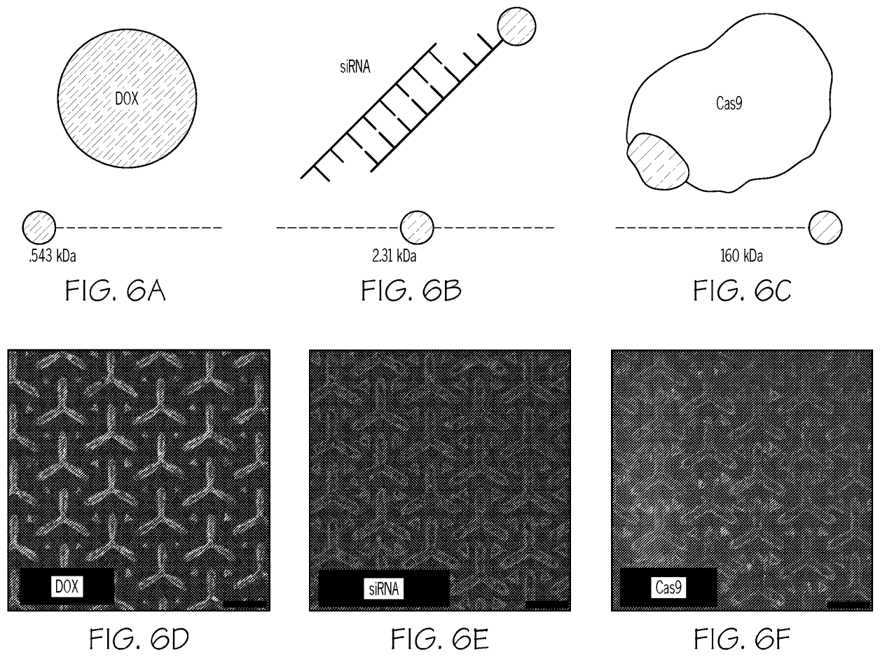

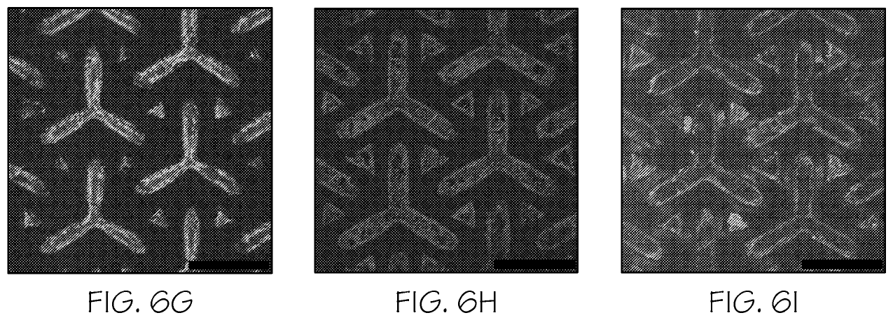

In this Example, Applicant explored the loading of siRNA (Cy3-labeled) and protein (Cas9-GFP), cargo of intriguing clinical interest, notable delivery challenges, and starkly different molecular properties (FIGS. 6A-6C). More fundamentally than the ability to load, Applicant intended to explore whether nanoscale photoporation was suitable for use with labile cargo without destruction due to localized heating.

For testing, Applicant returned to idealized solutions and exosome standards (COLO-1) and utilized previously optimized loading parameters for DOX. However, due to the lower concentrations of siRNA and Cas9, stock solutions of 1.25 μM were utilized, respectively, which were substituted for DOX in working solutions and used as input for magnetic microchips.

Furthermore, due to the minute quantities of siRNA and Cas9, Applicant was unable to resolve standard calibration curves for each as completed for DOX to quantify magnitude of loading. Instead, Applicant relied on on-chip confocal microscopy to resolve loading of cargo. Results from loading are shown in FIGS. 6D-6I, which shows successful localization of both siRNA and Cas9 upon micromagnets where exosomes are selectively captured.

### Example 1.5. Exotherm Photothermal Engineering System

EXOtherm was modularly constructed using a combination of custom 3D printed parts which include the housing and control spacing, and off-the-shelf components. 3D printed parts include the base, microchip holder, and thermoelectric cooler (TEC) holder. The base provides overall structure for the system and provides a housing for the permanent magnet (1″×1″×0.5″, N52, KJMagnetics). The microchip holder sits atop the base, is locked in place with magnetic fixturing, and has a space that allows for the alignment of a 3″×2″ glass slide, which was the substrate used for magnetic microchips. Once aligned, the magnetic microchip is also locked in place with magnetic fixturing. Atop the microchip holder, a holder for the TEC (Laird Thermal Systems, DA-044-12-02-00-00) is posited, which finely controls the distance between the LED (Luminus, CLM-22) and magnetic patterning to enable consistent irradiation. The LED optical output is controlled by a programmable power supply (Keysight 36155A), connected with interchangeable chassis mount resistors to protect against current spikes and enable high power operation.

To protect LED, and to enable operation beyond its specified max drive current, Applicant actively cooled the LED during pulsed operation. Further, to protect the user given high power operation, electrical connects from power supply are connected to an intermediate housing that provides electrical isolation.

### Example 1.6. Fabrication of Magnetic Microchips

Magnetic microchips were fabricated using a laser-based patterning and peel progress developed by Applicant. Briefly, components constituting a micromagnet composite (50:50 w/w Fc₃O₄ and PDMS) were manually mixed, desiccated, and blade coated upon a glass slide to desired thickness (KTQ-II adjustable fabricator, 10 μm precision). Due to the microscale thickness of ~35 μm, the composite can be rapidly cured on a hotplate at 150 °C (<10 minutes). Laser ablation of micromagnet composite is completed using a Technifor Laser Marking Machine (LW1, Gravotech) equipped with a 532-nm Diode Pump Solid State (DPSS) laser. Following laser ablation, the excess unwanted areas could easily be peeled away using tweezers, leaving free standing micromagnets. Finally, the fabricated micromagnets were mechanically stabilized using a solution-based dip-coating treatment with Al₂O₃, optimized to form a nanoscale thick coating (~100 nm), and a microwell placed above forming a fluidic cell.

To form microwells, 3D printed rings, designed to hold ~200 μL, were printed (Bambu PIP) followed by dipping the bottom of the well within PDMS (Sylgard 184, 10:1 base, curing agent) and aligning over pattern and bringing into contact, whereby the PDMS was cured on a hot plate (60 °C for 2 hours). Prior to use, an oxygen plasma treatment (29.8 W, 3 minutes, Herrick Plasma PDC-001) was performed to increase wettability.

### Example 1.7. Magnetic Nanoparticle Conjugation Preparation

Biotinylated anti-human antibodies (CD9, CD81, CD63) were purchased from BioLegend and used without further modification. Streptavidin-coated iron oxide nanoparticles (500 nm diameter) were purchased from Luna Nanotech, and prior to use, beads were washed twice within a magnetic stand with 1× PBS. Magnetic bead conjugates were prepared as described previously. Briefly, 20 μL stock antibody solution was combined with 175 μL magnetic beads and diluted to 400 AL with PBST (PBS+0.1% Tween-20), resulting in a concentration of 0.025 μg antibody/μL. Labeling was completed with shaking for 1.5 hours at room temperature, followed by washing unbound antibody twice within a magnetic rack with PBST and resuspending to 400 μL with PBST+0.1% BSA. Antibody conjugated magnetic nanoparticles were either used immediately for exosome capture or stored at 4 °C until use.

### Example 1.8. Magnetic Labeling of Exosomes

Exosome standards (COLO-1, Novus Biologics) were purchased and reconstituted according to the manufacturer's recommendations with molecular grade DI water at a concentration of 1 μg/μL. Magnetic labeling of exosomes was completed in batch-based preparations, such that a single batch could be used for each experiment and was scaled down and up as necessary. In a typical experiment, 5 μL of reconstituted exosome standard was added to 20 μL conjugate (6.66 μL of CD9, CD63, and CD81), and diluted to 500 AL with PBST+0.1% BSA. Labeling was completed with mixing (Argos Technologies RotoFlex Plus) for 3 hours at room temperature, followed by washing twice within a magnetic rank with PBST+0.1% BSA and resuspending in 1000 μL, yielding a final concentration of 0.5 μg/100 μL solution. For exosome labeling within plasma samples, plasma obtained from Dartmouth Health was thawed at 37 °C for 15 minutes. 200 μL of clinical plasma was incubated with 0.2 μg of antibody (included of an equal ratio of CD9, CD63, and CD81) for 2 hours at room temperature and used directly after for on-chip testing.

### Example 1.9. Doxorubicin Loading Within Exosomes

Doxorubicin was purchased from Sigma Aldrich as a power and kept as a stock concentration within 1× PBS at 1 mM, hidden from light, and stored at 4 °C. A standard curve was created to quantify doxorubicin concentration in solutions. Due to the ability to thoroughly wash exosomes upon magnetic capture chips, Applicant elected to use high concentrations of doxorubicin in solution for loading studies. Following magnetic tagging of exosomes, washing away excess antibody, and resuspending, a 200 μL working volume was aliquoted and doxorubicin added to a final concentration of 150 μM and briefly mixed by pipetting the solution up and down. Immediately after mixing, the sample was introduced into the magnetic microchip placed within its holder to enable magnetic patterning and subject to LED irradiation. Immediately following LED irradiation, samples were washed twice using PBST with 0.1% BSA, then removed from their holder enabling the release of captured exosomes, which was completed by resuspending them in 200 μL PBST with 0.1% BSA. For loading of exosomes directly from plasma samples, samples were used directly after conjugation. Once on-chip loading was performed, the exosomes were again thoroughly washed on-chip using PBST with 0.1% BSA before the microchip was removed and exosomes resuspended for subsequent processing.

### Example 1.10. Loading of Labile Cargo

Labile cargo including Cy3-labeled siRNA (Thermo, Silencer™ Cy™ 3-labeled Negative Control No. 1 siRNA) and Cas9-GFP (Sigma, CAS9GFPPRO), were purchased and reconstituted as necessary per manufacturer recommendations. Due to the significantly lower quantity of both reagents compared to doxorubicin, stock solutions of 25 μM were made. Loading testing followed an identical protocol to initial small molecule testing where COLO-1 exosome standards (1 μg) within a 200 μL working volume were aliquoted from the prepared stock. From here, Cy3-labeled siRNA or Cas9-GFP were added to the solution to a concentration of 1.25 μM, mixed briefly with a pipette to ensure homogenous incorporation, and added to the microchip. Following loading, an identical washing procedure used for doxorubicin was used prior to on-chip confocal imaging.

Example 1.11. Imaging of Exosome Capture and Cargo Loading Exosome visualization was completed using a DiD lipid membrane stain (Invitrogen, Ex./Em. 644/665 nm), prepared by mixing 25 mg of DiD in 4 mL of dimethyl sulfoxide. Staining of exosomes was completed with a 15-minute incubation at 37 °C, using 2.5 μL/1 μg of exosomes, followed by thoroughly washing of the sample five times using PBST with 0.1% BSA within a magnetic rack. DiD staining was completed following cargo loading for exosome standards and plasma samples alike, with exosomes that were removed from capture microchips. Imaging was completed with an Andor W1 Spinning Disc confocal microscope under brightfield and fluorescence using a 488 nm laser for excitation for doxorubicin and Cas 9-GFP, 532 nm excitation for Cy3-labed siRNA, and 637 nm excitation for DiD membrane dye. Images were processed within the NIS Elements Workstation or Fiji ImageJ. Processed plasma samples were finally reconstituted to a final volume of 200 μL with PBST with 0.1% BSA.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of loading at least one material with at least one active agent, said method comprising:
associating the material with a surface;
associating the active agent with the material;
photothermally heating the surface-associated material to result in the encapsulation of the active agent into the surface-associated material; and
dissociating the active agent-loaded material from the surface.

2. The method of claim 1, wherein the material is selected from the group consisting of particles, magnetic particles, nanoparticles, magnetic nanoparticles, polymeric particles, viral particles, virus-like particles, lipid-based particles, nanomaterials, magnetic nanomaterials, materials comprising a biomolecular membrane, exosomes, liposomes, cells, or combinations thereof.

3. The method of claim 1, wherein the material comprises exosomes.

4. The method of claim 1, wherein the material comprises cells, wherein the cells are selected from the group consisting of immune cells, T-cells, B-cells, or combinations thereof.

5. (canceled)

6. The method of claim 1, wherein the active agent is selected from the group consisting of drugs, small molecules, biologics, proteins, peptides, peptoids, nucleic acids, RNA, siRNA, mRNA, miRNA, DNA, genes, gene editing systems, clustered regularly interspaced short palindromic repeats (CRISPR)-Cas gene editing systems, image contrast agents, quantum dots, or combinations thereof.

7. The method of claim 1, wherein the surface is in the form of a membrane comprising a plurality of photosensitizers, wherein the plurality of photosensitizers are operational to induce localized heating of the membrane upon photothermal heating, and wherein the plurality of photosensitizers comprise magnetic nanoparticles.

8. (canceled)

9. The method of claim 1, wherein the surface comprises a patterned surface, wherein the patterned surface is operational for facilitating the association of the material with the surface, wherein the patterned surface comprises one or more shapes selected from the group consisting of squares, herringbones, Y-like shapes, X-like shapes, or combinations thereof.

10. (canceled)

11. The method of claim 1, wherein the surface comprises material binding agents operational for associating the material with the surface.

12. The method of claim 1, wherein the surface is in the form of a microfluidic channel, wherein the microfluidic channel comprises an inlet for receiving the material and the active agent, and an outlet for releasing the active agent-loaded material.

13. The method of claim 1, wherein the surface comprises a magnetic surface, wherein the magnetic surface is associated with a magnet operational to apply a magnetic field to the magnetic surface.

14-15. (canceled)

16. The method of claim 1, wherein the associating of the material with the surface comprises flowing the material through the surface.

17. The method of claim 1, wherein the surface comprises a magnetic surface.

18. The method of claim 1, wherein the surface comprises a magnetic surface, and wherein the method comprises:
capturing the material by magnetic beads to form a magnetic bead-material complex;
associating the magnetic beads with the magnetic surface;
applying a magnetic field to the magnetic surface to result in the immobilization of the magnetic beads on the magnetic surface;
associating the active agent with the material;
photothermally heating the surface-associated material to result in the encapsulation of the active agent into the surface-associated material; and
removing the magnetic field from the magnetic surface to result in the dissociation of the active agent-loaded material from the magnetic surface.

19. The method of claim 18, further comprising a step of dissociating the active-agent loaded material from the magnetic beads.

20. The method of claim 18, wherein the associating of the magnetic beads with the magnetic surface occurs after the capture of the material by the magnetic beads.

21. The method of claim 18, wherein the magnetic beads comprise magnetic particles and material binding agents on the magnetic particles, wherein capturing comprises capture of the material by the material binding agents.

22. The method of claim 1, wherein the photothermal heating comprises photothermally heating the surface, wherein the photothermal heating of the surface results in the heating of the material by the surface.

23. The method of claim 1, wherein the photothermal heating comprises light irradiation from a light source, and wherein the light source comprises a light emitting diode (LED).

24-27. (canceled)

28. The method of claim 1, further comprising a step of washing the surface, wherein the washing occurs prior to dissociating the active agent-loaded material from the surface.

29. The method of claim 1, wherein the material comprises T-cells, wherein the active agent comprises one or more genes encoding a T-cell receptor, and wherein the method is used to engineer chimeric antigen receptor T-cells.

30. The method of claim 1, wherein the material comprises exosomes, wherein the active agent comprises one or more drugs, and wherein the method is used to engineer therapeutic exosomes.

31. A system operable for loading a material with an active agent, said system comprising:
a surface operable to associate with the material; and
a photothermal heating source proximal to the surface and operable to heat the surface.

32. The system of claim 31, further comprising a magnet operable to apply a magnetic field to the surface, wherein the magnet is positioned below the surface.

33. (canceled)

34. The system of claim 31, wherein the photothermal heating source is positioned above the surface.

35. The system of claim 31, further comprising a cooling unit operable for cooling the photothermal heating source.

36. The system of claim 31, wherein the photothermal heating source comprises a light source operable to emit light irradiation, wherein the light source comprises a light emitting diode (LED).

37. (canceled)

38. The system of claim 31, wherein the surface is in the form of a membrane comprising a plurality of photosensitizers, wherein the plurality of photosensitizers comprise magnetic nanoparticles are operational to induce localized heating of the membrane upon photothermal heating.

39. (canceled)

40. The system of claim 31, wherein the surface comprises a patterned surface, wherein the patterned surface is operational for facilitating the association of the material with the surface, wherein the patterned surface comprises one or more shapes selected from the group consisting of squares, herringbones, Y-like shapes, X-like shapes, or combinations thereof.

41. (canceled)

42. The system of claim 31, wherein the surface comprises material binding agents operational for associating the material with the surface.

43. The system of claim 31, wherein the surface is in the form of a microfluidic channel, wherein the microfluidic channel comprises an inlet for receiving the material and the active agent, and an outlet for releasing the active agent-loaded material.

44. The system of claim 31, wherein the surface comprises a magnetic surface.
